(19) United States
(12) Patent Application Publication
(10) Pub. No.: US 2020/0268261 A1
(43) Pub. Date: Aug. 27, 2020

(54) VITAL DATA COLLECTION SYSTEM, VITAL DATA RELAY DEVICE, VITAL DATA RELAY SYSTEM, AND VITAL DATA COLLECTION METHOD

(71) Applicant: YOKOGAWA ELECTRIC CORPORATION, Tokyo (JP)

(72) Inventor: Daisuke IKEGAMI, Musashino-shi, Tokyo (JP)

(21) Appl. No.: 16/650,007

(22) PCT Filed: Aug. 2, 2018
(86) PCT No.: PCT/JP2018/029126

§ 371 Date: Mar. 24, 2020

(30) Foreign Application Priority Data
Sep. 29, 2017 (JP) 2017-189738

(51) Int. Cl. A61B 5/0205; A61B 5/00; H04W 4/38; H04W 4/70

(52) U.S. Cl. CPC A61B 5/02055; A61B 5/002; A61B 5/0024; A61B 2503/20; A61B 5/681; H04W 4/38; H04W 4/70; A61B 5/0022

(57) ABSTRACT

A vital data collection system is for collecting vital data of an actor acting in an environment not reached or not easily reached by radio waves from a mobile phone base station. The vital data collection system includes a sensor, a relay device, and a collection server. The sensor is carried by the actor and measures vital data of the actor. The relay device is carried by the actor, acquires the vital data of the actor measured by the sensor, and transmits the acquired vital data by low-power long-range wireless communication. The collection server collects, via a network, the vital data transmitted from the relay device.

### CROSS-REFERENCE TO RELATED APPLICATION

The present application claims priority to and the benefit of Japanese Patent Application No. 2017-189738 filed Sep. 29, 2017, the entire contents of which are incorporated herein by reference.

### TECHNICAL FIELD

The present disclosure relates to a vital data collection system, a vital data relay device, a vital data relay system, and a vital data collection method.

### BACKGROUND

In recent years, the Internet of Things (IoT) and Industrial Internet of Things (IIoT) have been attracting attention. The IoT and IIoT are technologies for connecting any and all things (hardware terminals in general, such as sensors, devices, and apparatuses) to the Internet, using sensors to collect data of these things on a cloud server, analyzing the collected data, and using the analysis results for specific purposes (providing feedback).

It is expected that such IoT and the like will be used not only in industry but in a wide variety of fields. For example, patent literature (PTL) 1 to 3 below disclose techniques for collecting vital data (such as biological information on body temperature, heart rate, and the like) on a cloud server. The physical condition of multiple people can easily be managed, for example, using the techniques disclosed in PTL 1 to 3.

### CITATION LIST

### Patent Literature

PTL 1: JP2015-514512A

PTL 2: JP2017-511164A

PTL 3: JP2016-167141A

### SUMMARY

### Technical Problem

The above-described techniques disclosed in PTL 1 to 3 could be applied to management of the physical condition of workers in a plant or other location, management of the work process, or the like. For example, a sensor could be attached to workers in a plant or the like, and vital data measured by the sensor could be uploaded to a cloud server. This would allow implementation of a system capable of managing the physical condition of the workers, managing the work process, and the like. In this system, the vital data measured by the sensor needs to be transmitted to the cloud server via a wireless (or partially wired) communication network. The conditions for wireless communication may not always be good, however, depending on the work environment.

A certain work environment may, for example, be an environment not reached or not easily reached by radio waves from a mobile phone base station (special environment), such as an environment outside of the mobile phone service area. Examples of special environments include environments that are distant from city centers, have few mobile phone base stations, and frequently involve work over a wide area (several kilometers to several tens of kilometers square), such as mines, mountainous areas, oceans, and construction sites.

One known method for transmitting data in such special environments is to install a plurality of relay devices between the sensor and the communication base station (or a gateway that can connect communicatively to the communication base station) and transfer the data to the plurality of relay devices. With this known method, however, it becomes necessary to install more and more relay devices, particularly as the work area is larger. This leads to high installation and operating costs for communication equipment.

In the aforementioned special environment, a satellite communication link with a wider area of use could be used instead of a mobile phone network, but this leads to high communication costs. A fixed communication network (wired communication) could be used instead of a mobile phone network, but in this case as well, installation and operating costs for communication equipment increase, particularly as the work environment extends over a larger area.

The present disclosure was conceived in light of these circumstances and aims to provide a vital data collection system, a vital data relay device, a vital data relay system, and a vital data collection method that can transmit data using a commercially available terminal while reducing costs, even in a special environment.

### Solution to Problem

A vital data collection system according to an embodiment is for collecting vital data of an actor acting in an environment not reached or not easily reached by radio waves from a mobile phone base station. The vital data collection system includes a sensor to be carried by the actor and configured to measure vital data of the actor; a relay device to be carried by the actor and configured to acquire the vital data of the actor measured by the sensor and to transmit the acquired vital data by low-power long-range wireless communication; and a collection server configured to collect, via a network, the vital data transmitted from the relay device. In this way, the vital data collection system can transmit vital data via a relay device and can thereby transmit data using a commercially available terminal while reducing costs, even in a special environment.

In an embodiment, the relay device may include a first relay device and a second relay device to be carried by the actor, the first relay device may be configured to acquire the vital data measured by the sensor, transmit the acquired vital data to the collection server via the mobile phone base station when wireless communication with the mobile phone base station is possible, and transmit the acquired vital data to the second relay device when wireless communication with the mobile phone base station is not possible, and the second relay device may be configured to transmit, to the collection server by the low-power long-range wireless communication, the vital data transmitted from the first relay device. In this way, by the relay device including the first relay device and the second relay device, the vital data is transmitted to the collection server via the second relay device when the first relay device cannot transmit the vital data to the collection server. The vital data collection system can thereby transmit data using a commercially available terminal while reducing costs, even in a special environment.

In an embodiment, the first relay device may be configured to transmit the acquired vital data by short-range wireless communication when wireless communication with the mobile phone base station is not possible, and the second relay device may be configured to receive the vital data transmitted from the first relay device by the short-range wireless communication. In this way, the vital data in the vital data collection system is transmitted from the first relay device to the second relay device by short-range wireless communication. The vital data collection system can thereby transmit data using a commercially available terminal while reducing costs, even in a special environment.

In an embodiment, the relay device may be configured to acquire, and transmit by the low-power long-range wireless communication, at least one of position information of the sensor, time information, and identification information of the actor or the sensor in addition to the vital data, and the collection server may be configured to collect the vital data transmitted from the relay device and at least one of the position information of the sensor, the time information, and the identification information of the actor or the sensor. The vital data collection system can thereby transmit more data, in addition to the vital data, in a special environment.

In an embodiment, the low-power long-range wireless communication may be wireless communication conforming to a LoRa® (LoRa is a registered trademark in Japan, other countries, or both) communication standard. The vital data collection system can thereby transmit data using LoRa.

A vital data relay device according to an embodiment is for relaying vital data of an actor carrying the vital data relay device and acting in an environment not reached or not easily reached by radio waves from a mobile phone base station, the vital data relay device including an acquisition interface configured to acquire vital data of the actor, the vital data being measured by a sensor, and a transmission interface configured to transmit, by low-power long-range wireless communication, the vital data acquired by the acquisition interface. In this way, vitals measured by the sensor are transmitted by the vital data relay device. The vital data relay device can thereby transmit data using a commercially available terminal while reducing costs, even in a special environment.

In an embodiment, the acquisition interface may be configured to receive the vital data when the vital data is transmitted by short-range wireless communication. In this way, the vital data is relayed by short-range wireless communication. The vital data relay collection system can thereby transmit data using a commercially available terminal while reducing costs, even in a special environment.

In an embodiment, the acquisition interface may be configured to acquire at least one of position information of the sensor, time information, and identification information of the actor or the sensor in addition to the vital data, and the transmission interface may be configured to transmit the vital data acquired by the acquisition interface and at least one of the position information of the sensor, the time information, and the identification information of the actor or the sensor. The vital data relay device can thereby relay more data, in addition to the vital data, in a special environment.

A vital data relay system according to an embodiment includes a first relay device and a second relay device to be carried by an actor acting in an environment not reached or not easily reached by radio waves from a mobile phone base station. The first relay device is configured to acquire vital data measured by a sensor, transmit the acquired vital data via the mobile phone base station when wireless communication with the mobile phone base station is possible, and transmit the acquired vital data to the second relay device when wireless communication with the mobile phone base station is not possible, and the second relay device is configured to transmit, by low-power long-range wireless communication, the vital data transmitted from the first relay device. In this way, the vital data relay system can transmit vital data via a relay device and can thereby transmit data using a commercially available terminal while reducing costs, even in a special environment. The vital data relay system can also transmit the vital data via the second relay device when the first relay device cannot transmit the vital data to the collection server.

In an embodiment, the first relay device may be configured to transmit the acquired vital data by short-range wireless communication when wireless communication with the mobile phone base station is not possible, and the second relay device may be configured to receive the vital data transmitted from the first relay device by the short-range wireless communication. In this way, the vital data in the vital data collection system is transmitted from the first relay device to the second relay device by short-range wireless communication. The vital data collection system can thereby transmit data using a commercially available terminal while reducing costs, even in a special environment.

In an embodiment, the first relay device and the second relay device may be configured to transmit at least one of position information of the sensor, time information, and identification information of the actor or the sensor in addition to the vital data. The vital data relay system can thereby transmit more data, in addition to the vital data, in a special environment.

A vital data collection method according to an embodiment is for collecting vital data of an actor acting in an environment not reached or not easily reached by radio waves from a mobile phone base station, the vital data collection method including measuring vital data of the actor, using a sensor carried by the actor, acquiring, using a relay device carried by the actor, the vital data of the actor measured by the sensor, performing first transmission, using the relay device, to transmit the acquired vital data to a collection server by low-power long-range wireless communication, and collecting via a network, using the collection server, the vital data transmitted from the relay device. In this way, the vital data collection method allows transmission of vital data via a relay device and thereby allows transmission of data using a commercially available terminal while reducing costs, even in a special environment.

In an embodiment, the vital data collection method may further include performing second transmission, using the relay device, to transmit the acquired vital data to the collection server via the mobile phone base station instead of by the low-power long-range wireless communication when wireless communication with the mobile phone base station is possible. By inclusion of the second transmission, the vital data is thus transmitted to the collection server from the relay device via the mobile phone base station when wireless communication between the relay device and the mobile phone base station is possible. The vital data collection system can thereby transmit data using a commercially available terminal while reducing costs, even in a special environment.

In an embodiment, the performing first transmission and the performing second transmission may include transmitting the vital data acquired in the acquiring and at least one of position information of the sensor, time information, and identification information of the actor or the sensor, and the collecting may include collecting the vital data transmitted from the relay device and at least one of the position information of the sensor, the time information, and the identification information of the actor or the sensor. The vital data collection method can thereby transmit more data, in addition to the vital data, in a special environment.

### Advantageous Effect

A vital data collection system, a vital data relay device, a vital data relay system, and a vital data collection method according to the present disclosure can transmit data using a commercially available terminal while reducing costs, even in a special environment.

### BRIEF DESCRIPTION OF THE DRAWINGS

In the accompanying drawings:

FIG. 1 illustrates the overall configuration of a vital data collection system according to an embodiment;

FIG. 2 schematically illustrates the hardware configuration of a communication converter router of the vital data collection system according to an embodiment;

FIG. 3 is a block diagram illustrating the functional configuration of the communication converter router in the vital data collection system according to an embodiment;

FIG. 4A schematically illustrates an example of a screen displayed on a smartphone in the vital data collection system according to an embodiment;

FIG. 4B schematically illustrates an example of a screen displayed on a smartphone in the vital data collection system according to an embodiment;

FIG. 4C schematically illustrates an example of a screen displayed on a smartphone in the vital data collection system according to an embodiment;

FIG. 4D schematically illustrates an example of a screen displayed on a smartphone in the vital data collection system according to an embodiment;

FIG. 5A schematically illustrates an example of a screen displayed on a health watch in the vital data collection system according to an embodiment;

FIG. 5B schematically illustrates an example of a screen displayed on a health watch in the vital data collection system according to an embodiment;

FIG. 5C schematically illustrates an example of a screen displayed on a health watch in the vital data collection system according to an embodiment;

FIG. 5D schematically illustrates an example of a screen displayed on a health watch in the vital data collection system according to an embodiment;

FIG. 6 schematically illustrates an example of a system administrator screen generated by a cloud server of the vital data collection system according to an embodiment;

FIG. 7 schematically illustrates an example of a system administrator screen generated by a cloud server of the vital data collection system according to an embodiment;

FIG. 8 is a flowchart illustrating an example of operations of the communication converter router in the vital data collection system according to an embodiment; and

FIG. 9 is a flowchart illustrating an example of operations of a relay device in the vital data collection system according to an embodiment.

### DETAILED DESCRIPTION

### Embodiment

An embodiment is described below.

[Overall Configuration of Vital Data Collection System]

The overall configuration of a vital data collection system 1 is described below with reference to the drawings.

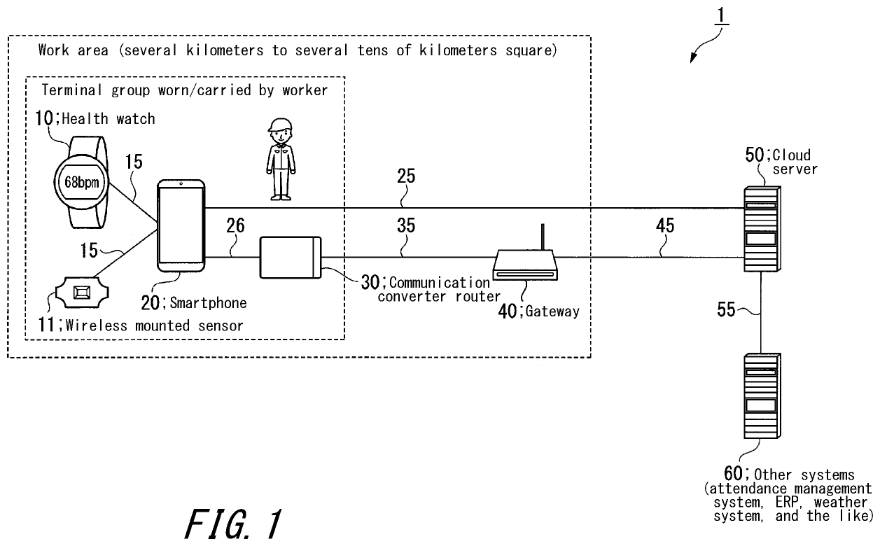

FIG. 1 illustrates the overall configuration of the vital data collection system 1 according to an embodiment. As illustrated, the vital data collection system 1 includes a health watch 10, a wireless mounted sensor 11, a short-range wireless communication path 15, a smartphone 20, a mobile phone network 25, a short-range wireless communication path 26, a communication converter router 30, a low-power long-range wireless communication path 35, a gateway 40, a mobile phone network 45, a cloud server 50, a communication network 55, and other systems 60.

The vital data collection system 1 according to the present embodiment is a system, implemented by cloud computing, mainly for recognition of the physical condition of workers working in a special environment, attendance management of the workers, and work process management in a special environment.

In FIG. 1, only a terminal group worn/carried by one worker is depicted. A plurality of workers are actually present in the work area, however, and each worker wears/carries the aforementioned terminal group.

The cloud computing referred to here matches the definitions (recommended by the U.S. National Institute of Standards and Technology) listed in the documents specified by the following uniform resource locators (URL), for example.

http://nvlpubs.nist.gov/nistpubs/Legacy/SP/nistspecialpublication800-145.pdf

https://www.ipa.go.jp/files/000025366.pdf

The health watch 10 is a wristwatch-type wearable device worn on the wrist of a worker working in the special environment. The health watch 10 includes a variety of sensors that can be used for vital sign sensing with respect to the worker. The vital sign sensing referred to here is a technique for using various sensors to measure body temperature, pulse rate, blood pressure, amount of exercise, posture, heart rate, electrocardiogram, blood oxygen concentration, and the like.

Other than sensors used for vital sign sensing, the health watch 10 also includes components such as an acceleration sensor and a receiver for receiving radio waves of a Global Navigation Satellite System (GNSS), such as a Global Positioning System (GPS) or a Global Navigation Satellite System (GLONASS). The health watch 10 can also measure the movement, position, and the like of the worker.

The health watch 10 includes a communication function and can communicatively connect to the smartphone 20 via the short-range wireless communication path 15. For example, the health watch 10 transmits measurement data, which includes position information; time information data; and identification information of a worker or the health watch 10 to the smartphone 20.

As long as the health watch 10 is a wearable device capable of performing the aforementioned vital sensing and capable of communicating with the smartphone 20, the health watch 10 need not be a wristwatch-type device.

The wireless mounted sensor 11 is a sensor worn by a worker working in a special environment. The wireless mounted sensor 11 is, for example, attached directly to the worker's clothes, or to the worker's skin, by a wearable band, sticker, or the like. The wireless mounted sensor 11 performs vital sign sensing or the like with respect to the worker, like the health watch 10. The wireless mounted sensor 11 also has a communication function, like the health watch 10, is capable of communicatively connecting to the smartphone 20 via the short-range wireless communication path 15, and transmits measurement data to the smartphone.

One worker may, for example, wear a plurality of different types of wireless mounted sensors 11. The wireless mounted sensor 11 is a positioning device that supplements the functions of the health watch 10 and measures vital signs or the like that cannot be measured by the health watch 10. The vital data collection system 1 may therefore be configured without the wireless mounted sensor 11 if the desired data can all be acquired with only the health watch 10.

In the explanation below, the health watch 10 and the wireless mounted sensor 11 are collectively referred to as the “health watch 10 and the like”.

In the explanation below, the various measurement data measured by the health watch 10 and the like (specifically, body temperature, pulse rate, blood pressure, amount of exercise, posture, heart rate, electrocardiogram, blood oxygen concentration, and the like) is collectively referred to as “vital data”.

The short-range wireless communication path 15 is a communication path for short-range wireless communication. The short-range wireless communication referred to here is, for example, Bluetooth®, wireless fidelity, i.e. Wi-Fi®, or the like (Bluetooth and Wi-Fi are registered trademarks in Japan, other countries, or both).

The smartphone 20 (first relay device) is a handheld information terminal carried by a worker working in the special environment. The smartphone 20 can pair with the health watch 10 and the like and acquire information from the health watch 10 and the like via the short-range wireless communication path 15. Such information includes the vital data, the position information of the health watch 10 and the like, time information, and identification information (ID) of the worker or of the health watch 10 and the like. The smartphone 20 can communicatively connect to the cloud server 50 via the mobile phone network 25. The smartphone 20 can communicatively connect to the communication converter router 30 via the short-range wireless communication path 26.

When the smartphone 20 can communicatively connect via the mobile phone network 25 (i.e. when the smartphone 20 can communicate wirelessly with a mobile phone base station), the smartphone 20 transmits the vital data acquired from the health watch 10 and the like to the cloud server 50 via the mobile phone network 25 (via a mobile phone base station).

Conversely, when the smartphone 20 cannot communicatively connect via the mobile phone network 25 (i.e. when the smartphone 20 cannot communicate wirelessly with a mobile phone base station), the smartphone 20 transmits the vital data acquired from the health watch 10 and the like to the communication converter router 30 via the short-range wireless communication path 26.

Together with the vital data, at least one of the aforementioned position information, time information, and identification information may be transmitted from the smartphone 20 to the cloud server 50 or the communication converter router 30. The “vital data” is referred to below as being transmitted from the smartphone 20, and the “vital data” as being collected on the cloud server 50. However, to avoid a redundant description, it should be noted that this refers not only to the case of the vital data alone being transmitted but also includes the case of the vital data and at least one of the aforementioned position information, time information, and identification information being transmitted and collected.

The mobile phone network 25 is a communication network for mobile phone communication. As referred to here, mobile phone communication is wireless communication conforming to communication standards such as long term evolution (LTE) or the 3^{rd} generation mobile communication system (3G).

The short-range wireless communication path 26 is a communication path for short-range wireless communication. As referred to here, short-range wireless communication is wireless communication conforming to a communication standard such as Bluetooth® or Wi-Fi®.

The communication converter router 30 (second relay device) can communicatively connect to the smartphone 20 via the short-range wireless communication path 26. The communication converter router 30 acquires vital data transmitted from the smartphone 20 via the short-range wireless communication path 26.

The communication converter router 30 can communicatively connect to the gateway 40 via the low-power long-range wireless communication path 35. The communication converter router 30 transmits the vital data acquired from the smartphone 20 to the gateway 40 via the low-power long-range wireless communication path 35.

In other words, the communication converter router 30 is a vital data relay device that uses low-power long-range wireless communication to transmit the vital data acquired via short-range wireless communication.

The low-power long-range wireless communication path 35 is a communication path over which long-range wireless communication is possible at low power, i.e. a low-power wide-area network (LPWAN). As referred to here, low-power long-range wireless communication is wireless communication conforming to a communication standard such LoRa®.

As illustrated in FIG. 1, a worker in a special environment works while wearing the health watch 10 and the wireless mounted sensor 11 and carrying the smartphone 20 and the communication converter router 30.

The gateway 40 can communicatively connect to the communication converter router 30 via the low-power long-range wireless communication path 35. The gateway 40 acquires the vital data, transmitted from the communication converter router 30, via the low-power long-range wireless communication path 35.

The gateway 40 can communicatively connect to the cloud server 50 via the mobile phone network 45. The gateway 40 transmits the vital data acquired from the communication converter router 30 to the cloud server 50 via the mobile phone network 45.

In other words, the gateway 40 uses mobile phone communication to transmit the vital data acquired by low-power long-range wireless communication.

The gateway 40 is installed at a point within the work area allowing communicative connection via the mobile phone network 45 (i.e. a point within the mobile phone service area). The communication converter router 30 of a worker located at a point outside the mobile phone service area transmits the vital data, via the low-power long-range wireless communication path 35, to the gateway 40, which is capable of mobile phone communication. The communication converter router 30 thereby transmits the vital data to the cloud server via the gateway 40.

The work area may, for example, be a wide area of several kilometers to several tens of kilometers square, and the distance from the worker to the gateway 40 may be several tens of kilometers. For this reason, low-power long-range wireless communication, such as LoRa®, that allows low-power communication even over several tens of kilometers without the use of a base station, relay device, or the like is used for the communication between the terminal carried by the worker (communication converter router 30) and the gateway 40.

The mobile phone network 45 is a communication network for mobile phone communication. As referred to here, mobile phone communication is wireless communication conforming to a communication standard such as LTE or 3G.

The cloud server 50 acquires the vital data, transmitted from the smartphone 20, via the mobile phone network 25. The cloud server 50 also acquires the vital data, transmitted from the gateway 40, via the mobile phone network 45 and manages the vital data.

The cloud server 50 can communicatively connect to the other systems 60 via the communication network 55. The cloud server 50 acquires various data, transmitted from the other systems 60, via the communication network 55.

The communication network 55 is, for example, configured by the Internet, any of various closed networks (such as a dedicated line, a virtual private network (VPN), or the like), or a combination of these communication networks. The communication network 55 may be a wired communication network, or part or all of the communication network 55 may be a wireless communication network.

The other systems 60 are, for example, an attendance management system, an enterprise resource planning (ERP) system, a weather system, and the like. In the present disclosure, these systems are collectively referred to as “other systems 60”.

The cloud server 50 provides various cloud services using the vital data acquired from the smartphone 20 or the communication converter router 30 and various data acquired from the other systems 60 (such as attendance management data, ERP data, weather data, and the like).

The “cloud services” referred to here are, for example, a worker health management service and a work process management service provided to an administrator who manages workers working in a special environment. Details of these cloud services are provided below.

[Hardware Configuration of Communication Converter Router]

The hardware configuration of the communication converter router 30 is described below with reference to the drawings.

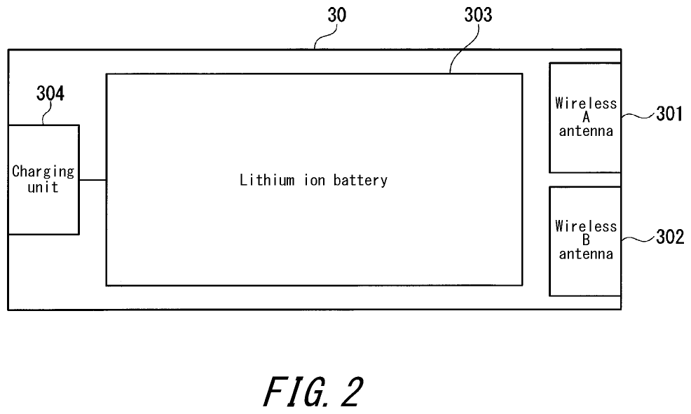

FIG. 2 schematically illustrates the hardware configuration of the communication converter router 30 of the vital data collection system 1 according to an embodiment. As illustrated, the communication converter router 30 includes a wireless A antenna 301, a wireless B antenna 302, a lithium ion battery 303, and a charging unit 304.

The wireless A antenna 301 is an antenna for transmitting and receiving radio waves of short-range wireless communication (such as Bluetooth® or Wi-Fi®).

The wireless B antenna 302 is an antenna for transmitting and receiving radio waves of low-power long-range wireless communication (such as LoRa®). The wireless A antenna 301 and the wireless B antenna 302 are preferably antennas embedded within the housing of the communication converter router 30 so as not to impede the work performed by the worker carrying the communication converter router 30.

The lithium ion battery 303 is a secondary cell that is capable of charging and discharging by lithium ions moving between the positive electrode and the negative electrode and that can be used repeatedly (e.g. capable of being charged approximately 1,000 times). The lithium ion battery 303 is, for example, a lightweight, thin lithium ion battery capable of repeated use throughout one workday (such as eight hours) of the worker. The lithium ion battery 303 preferably includes an overcharge prevention function. Another secondary cell, such as a magnesium battery, may be used instead of a lithium ion battery.

The charging unit 304 is configured to include a power supply apparatus (not illustrated) that creates a DC (direct current) current for charging from a commercial power supply and a charging control circuit (not illustrated) that controls charging of the lithium ion battery 303. To facilitate charging by the worker in various environments, the charging unit 304 preferably is configured to allow charging with a general-purpose charging method similar to the charging method of the smartphone 20, for example. The general-purpose charging method is, for example, a charging method for using a commercially available universal serial bus (USB) cable or commercially available alternating current (AC) adapter to input alternating current (AC) power from a commercial power supply.

So as not to impede the work performed by the worker, the communication converter router 30 is preferably a device that is compact (for example, less than 1 cm thick so as to fit in the worker's pocket) and lightweight (for example, less than 500 g).

The communication converter router 30 is used in special environments such as mines, mountainous areas, oceans, and construction sites, and therefore is preferably configured to be waterproof and dustproof (for example, in compliance with IP67 prescribed by the International Electrotechnical Commission (IEC)). When also used in areas designated as explosion proof, the communication converter router 30 is preferably also explosion proof.

The power button of the communication converter router 30 is, for example, preferably located at a difficult-to-press position on the side of the communication converter router 30 so that the power button is not pressed unintentionally by contact with another object during work. A light emitting diode (LED) lamp displaying the communication state (or the operating state of the communication converter router 30), for example, is also preferably disposed on the side of the communication converter router 30 so that the worker can easily confirm the communication state while working.

[Functional Configuration of Communication Converter Router]

The functional configuration of the communication converter router 30 is described below with reference to the drawings.

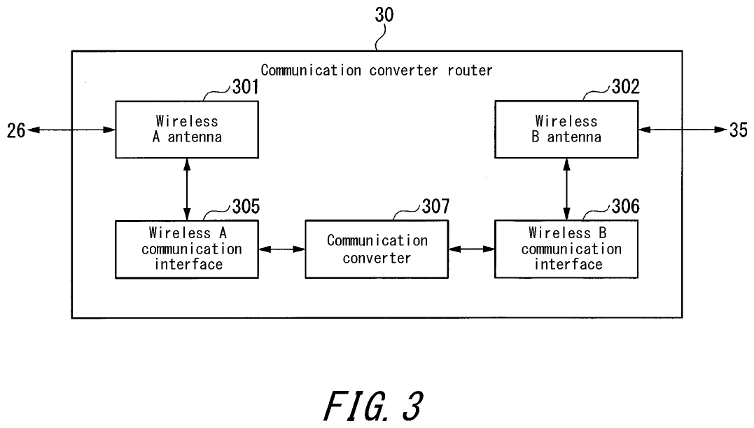

FIG. 3 is a block diagram illustrating the functional configuration of the communication converter router 30 in the vital data collection system 1 according to an embodiment. As illustrated, the communication converter router 30 of the vital data collection system 1 includes the wireless A antenna 301, the wireless B antenna 302, a wireless A communication interface 305, a wireless B communication interface 306, and a communication converter 307.

The wireless A antenna 301 transmits and receives radio waves for short-range wireless communication in response to control by the wireless A communication interface 305. As described above, the short-range wireless communication is Bluetooth®, Wi-Fi®, or the like.

The wireless B antenna 302 transmits and receives radio waves for low-power long-range wireless communication in response to control by the wireless B communication interface 306. The low-power long-range wireless communication is, for example, the above-described LoRa®. Alternatively, the low-power long-range wireless communication may, for example, be Sigfox®, narrow band-IoT (NB-IoT), ZigBee®, Z-Wave®, or the like (Sigfox, ZigBee, and Z-Wave are registered trademarks in Japan, other countries, or both).

The wireless A communication interface 305 controls the transmission and reception of signals during short-range wireless communication with the smartphone 20 via the wireless A antenna 301.

The wireless B communication interface 306 controls the transmission and reception of signals during low-power long-range wireless communication with the gateway 40 via the wireless B antenna 302.

The communication converter 307 acquires a short-range wireless communication signal from the wireless A communication interface 305, converts the acquired signal to a low-power long-range wireless communication signal, and outputs the converted signal to the wireless B communication interface 306. The communication converter 307 also acquires a low-power long-range wireless communication signal from the wireless B communication interface 306, converts the acquired signal to a short-range wireless communication signal, and outputs the converted signal to the wireless A communication interface 305.

[Use Process by Worker]

An example work process by a worker in the vital data collection system 1 is now described.

In advance, a worker (or administrator) accesses a cloud service on the cloud server 50 in an environment capable of communicably connecting to the Internet and sets a login identifier (ID) and a password. The login ID may, for example, be an e-mail address, a mobile phone number, or the like.

From the smartphone 20 used for work, a worker accesses the website of an operating system (OS) vendor (such as Apple or Google) or the website of a dedicated application store. The worker downloads a dedicated application for the cloud service onto the smartphone 20 (or acquires the dedicated application delivered through mobile device management application software) and installs the dedicated application.

The worker sets the health watch 10 and the smartphone 20 to pairing mode. The health watch 10 and the smartphone 20 are thereby paired (an inter-device association is formed) by wireless communication (such as Bluetooth®), allowing data to be exchanged between the health watch 10 and the smartphone 20.

The worker launches the installed dedicated application on the smartphone 20, logs in, and taps a “device registration button” displayed on the dedicated application, for example, to register the health watch 10 as one of the sensors to be used in the cloud service (a sensor targeted for vital data collection). After registration, an e-mail including information such as a guide for starting to use the cloud service is transmitted by the cloud service to the e-mail address of the worker and the e-mail address of the administrator.

The worker wears the health watch 10 on the wrist while the health watch 10 and the smartphone 20 are paired and makes initial settings in the dedicated application on the smartphone 20 to activate a fall detection function and an anomaly detection function. The fall detection function referred to here is a function for the cloud service to detect that the worker has fallen. The anomaly detection function referred to here is a function for the cloud service to detect that the worker is not acting (no change in action). An acceleration sensor is used for the initial settings. The initial settings are made so that the state when the worker has lowered his arm in a natural position is taken as a reference, and a fall or anomaly is judged to have occurred when a change in angle from the reference continues for a certain time.

At the start of work in the work area, the worker provides operation input on the health watch 10 to indicate the start of work (for example, the worker displays an operation menu on the screen of the health watch 10, operates a button provided on the side of the health watch 10, and selects “start work” from the operation menu). Upon operation input indicating the start of work being provided on the health watch 10, information indicating the work start time is transmitted to the cloud service on the cloud server 50 via the smartphone 20.

After operation input indicating the start of work is provided, data indicating measured values measured by the health watch 10 is transmitted to the cloud service on the cloud server 50 via the smartphone 20 at set intervals (such as an interval selected from among once every 1 second, 10 seconds, 30 seconds, 1 minute, or 5 minutes, or the initial default of once every 1 minute when no interval is set). The data indicating various measured values is, for example, vital data such as body temperature, pulse rate, blood pressure, amount of exercise, posture, acceleration, and position.

The worker provides operation input indicating the start of a break when taking a break from working in the work area (for example, the worker displays the operation menu on the screen of the health watch 10, operates the button provided on the side of the health watch 10, and selects “start break” from the operation menu). By this operation, all of the vital data of the worker during the break (or just specific vital data related to privacy, such as position information) is not collected on the cloud server. The worker's privacy is thereby protected.

At the end of the break, the worker provides operation input indicating the end of the break (for example, the worker displays the operation menu on the screen of the health watch 10, operates the button provided on the side of the health watch 10, and selects “end break” from the operation menu). With this operation, the temporarily suspended collection of vital data by the health watch 10 begins again.

At the end of work in the work area, the worker provides operation input on the health watch 10 to indicate the end of work (for example, the worker displays an operation menu on the screen of the health watch 10, operates a button provided on the side of the health watch 10, and selects “end work” from the operation menu). Upon operation input indicating the end of work being provided on the health watch 10, information indicating the work end time is transmitted to the cloud service on the cloud server 50 via the smartphone 20. A configuration may be adopted so that even when the worker forgets to provide operation input indicating the end of work, the work end time can be registered with the cloud service after the fact.

The provision of operation input indicating the end of work suspends the collection of all vital data of the worker by the health watch 10 at fixed intervals.

By the cloud server 50 working in conjunction with the other systems 60, such as a weather system, the cloud service may be configured to transmit a message related to weather information to the smartphone 20 of the worker (or to the health watch 10 connected wirelessly via the smartphone 20 of the worker). The message related to weather information as referred to here is, for example, a notification message indicating an instruction or recommendation for the worker, such as “Potential thunderstorm in 10 minutes. Stop working”. Based on the weather information and information transmitted from the health watch 10 and the like (such as information indicating the work start time or the physical condition), the cloud service may be configured to transmit a message to the smartphone 20 with a recommendation such as that the worker drink water or take a break.

[Examples of Use Screen Displayed on Smartphone]

Examples of use screens, displayed on the smartphone 20, for the dedicated application of the cloud service are described below.

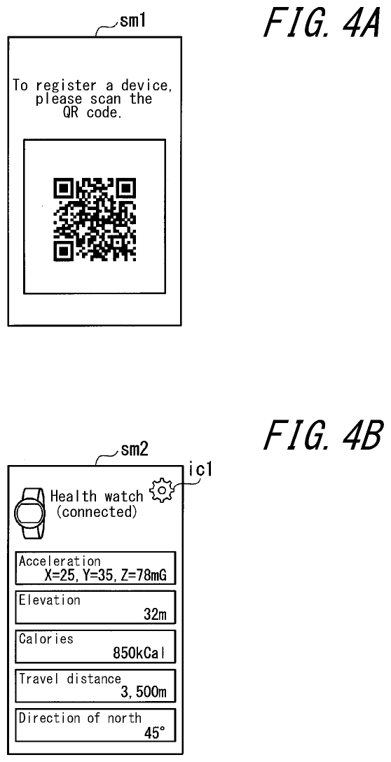

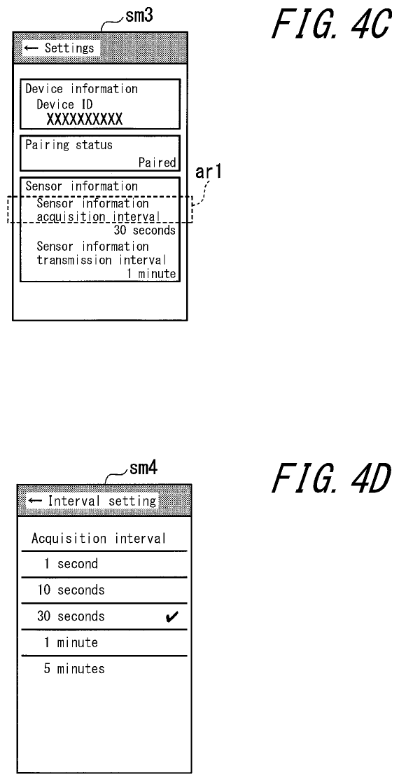

FIGS. 4A through 4D schematically illustrate examples of screens displayed on the smartphone 20 in the vital data collection system 1 according to an embodiment.

The screen sm1 illustrated in FIG. 4A is a device registration screen of the dedicated application and is displayed on the smartphone 20 at the time of device registration. The worker reads a QR code® (QR code is a registered trademark in Japan, other countries, or both) attached to the health watch 10 (or a QR code® displayed on the screen of the health watch 10) using the camera provided in the smartphone 20 that the worker uses for work. Upon the QR code® being read, information indicating the targeted health watch and a “device registration button” are displayed on the screen of the dedicated application, for example. The worker touches the device registration button to register the health watch 10 as one of the sensors used in the cloud service.

Other methods for registering the health watch 10 with the cloud service besides the aforementioned registration using a QR code® include pairing the smartphone 20 and the health watch 10 by short-range wireless communication, such as Bluetooth® or Wi-Fi®.

The screen sm2 illustrated in FIG. 4B is a sensor information display screen of the dedicated application and is displayed on the smartphone 20 when an operation to display the information of the registered sensor is performed after the aforementioned device registration. As illustrated, the name and image of the health watch 10 for which the aforementioned device registration was performed, along with text such as “connected” to indicate that the health watch 10 and the smartphone 20 are communicatively connected, are displayed on the screen sm2. Information indicating the vital data of the worker and the state of the health watch 10, such as “acceleration”, “elevation”, “calories”, “travel distance”, and “direction of north” are displayed on the screen sm2. An icon ic1 indicating a setting button is also displayed in the upper right area of the screen sm2.

The screen sm3 illustrated in FIG. 4C is a settings screen displayed when the area of the icon ic1 displayed on the screen sm2 illustrated in FIG. 4B is tapped. As illustrated, device information indicating the device ID of the aforementioned registered device (i.e. an identifier identifying the health watch 10 registered as a device) is displayed on the screen sm3. Information indicating the pairing state between the smartphone 20 and the device (health watch 10) corresponding to the device ID displayed in the device information is also displayed on the screen sm3, as illustrated.

Sensor information indicating a sensor information acquisition interval and a sensor information transmission interval is also displayed on the screen sm3, as illustrated. The sensor information acquisition interval indicates the interval at which the smartphone 20 periodically acquires vital data, indicating the measured values measured by the sensor (health watch 10), from the sensor. The sensor information transmission interval indicates the interval at which the smartphone 20 periodically transmits the vital data acquired from the sensor (health watch 10) to the cloud server 50.

The screen sm4 illustrated in FIG. 4D is a settings screen displayed when an area ar1 is tapped. The area ar1 is indicated as “sensor information acquisition interval” in the display area of the “sensor information” in the screen sm3 of FIG. 4C. This settings screen is for the worker to set the interval at which the smartphone 20 acquires the vital data from the sensor (health watch 10). As illustrated, a check mark is displayed in the area to the right of the area where “30 seconds” is displayed on the screen sm4. This indicates that the smartphone 20 is set to acquire the vital data from the sensor at thirty second intervals. The worker can set the sensor information acquisition interval to the desired interval by tapping the regions displayed as “1 second”, “10 seconds”, “30 seconds”, “1 minute”, and “5 minutes”.

[Examples of Use Screen Displayed on Health Watch]

The following describes examples of use screens displayed on the health watch 10.

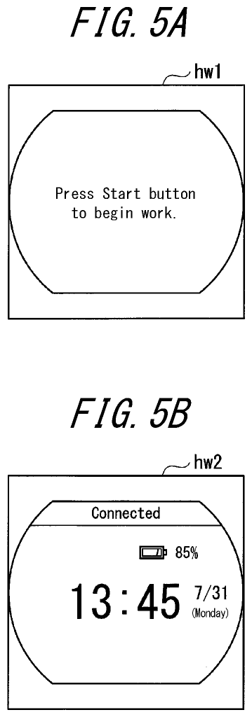

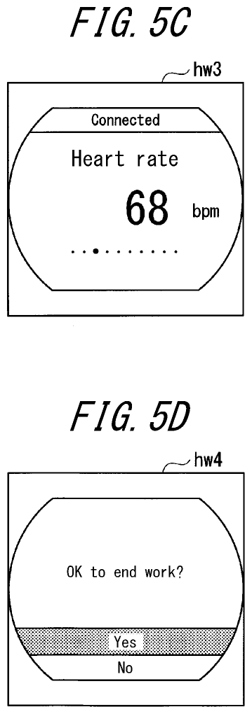

FIGS. 5A through 5D schematically illustrate examples of screens displayed on the health watch 10 in the vital data collection system 1 according to an embodiment.

The screen hw1 illustrated in FIG. 5A is a work start confirmation screen displayed on the health watch 10 when the health watch 10 and the smartphone 20 are wirelessly paired. As illustrated, the text “press Start button to start work”, encouraging the worker to confirm the start of work, is displayed on the screen hw1. When the worker presses a start button (not illustrated), for example, provided on the side of the health watch 10 (provides operation input indicating the start of work), then information indicating the work start time is transmitted to the cloud service on the cloud server 50 via the smartphone 20, and periodic vital data collection by the health watch 10 begins.

The screen hw2 illustrated in FIG. 5B is a watch screen displayed when the start button (not illustrated) is pressed (operation input indicating the start of work is provided) while the screen hw1 in FIG. 5A is displayed. In other words, the screen hw2 is a default screen when vital data is being periodically collected by the health watch 10 (while the worker is working). As illustrated, the screen hw2 displays not only the current time but also the date, the text “Connected” indicating that the health watch 10 and the smartphone 20 are paired, the battery level of the health watch 10, and the like.

The screen hw3 illustrated in FIG. 5C is a sensor information display screen displayed when the start button (not illustrated), for example, is pressed while the screen hw2 in FIG. 5B is displayed. As illustrated, the screen hw3 displays not only information indicating the heart rate of the worker measured by the health watch 10, but also the text “Connected” indicating that the health watch 10 and the smartphone 20 are paired. When the worker presses a Next button (not illustrated) provided on the side of the health watch 10, for example, while the screen hw3 is displayed, the display switches to vital data other than the heart rate (such as body temperature or blood pressure).

When the worker presses a back button (not illustrated) provided on the side of the health watch 10, for example, while the screen hw3 is displayed, the screen hw2 (watch screen, i.e. default screen) illustrated in FIG. 5B is displayed again.

The screen hw4 illustrated in FIG. 5D is a work end confirmation screen displayed when the back button (not illustrated), for example, is pressed while the screen hw2 illustrated in FIG. 5B is displayed. As illustrated, the text “OK to end work?” encouraging the worker to confirm the end of work along with the prompts “yes” and “no” are displayed on the screen hw4. When the worker provides operation input selecting “yes” (operation input indicating to end work) while the screen hw4 is displayed, the information indicating the work end time is transmitted to the cloud service on the cloud server 50 via the smartphone 20, and periodic collection of vital data by the health watch 10 ends.

[Examples of System Administrator Screen]

Examples of a system administrator screen provided to an administrator (such as a site supervisor or a remote administrator) by the cloud service operating on the cloud server 50 are described below with reference to the drawings.

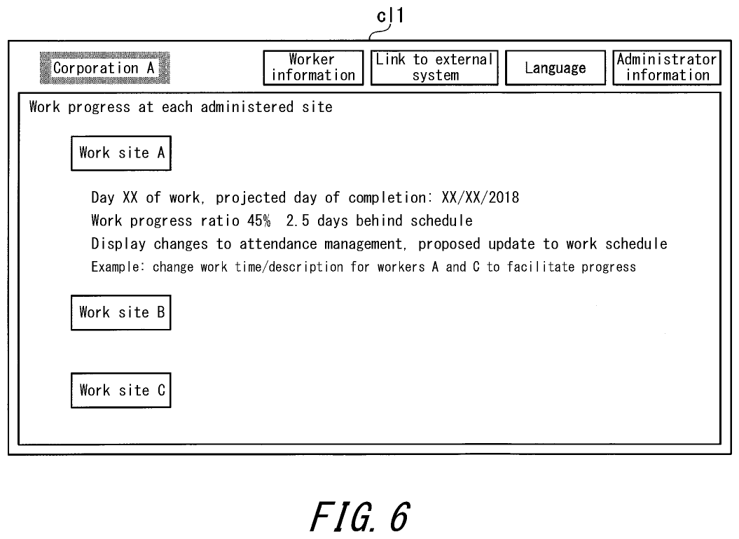

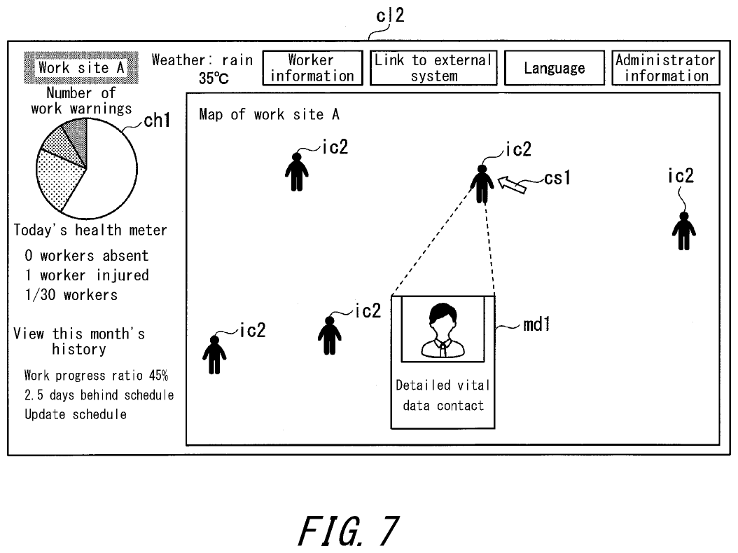

FIGS. 6 and 7 schematically illustrate examples of system administrator screens generated by the cloud server 50 of the vital data collection system 1 according to an embodiment.

A system administrator screen cl1 illustrated in FIG. 6 is a work progress management screen referred to by the administrator to confirm the progress of work at each site over which the administrator has authority (administered site). The system administrator screen cl1 is, for example, the initial screen (default screen) displayed when the administrator logs into the cloud service.

As illustrated, information related to the work progress, such as the number of days from the start of the work process, the anticipated end date of the work process, the current work progress ratio, and the current number of days that work is behind schedule, is displayed for each work site (work area) on the system administrator screen cl1. Proposed changes to attendance management for workers, a proposed update to the work schedule, and the like are also displayed for the work process on the system administrator screen cl1.

With the system administrator screen cl1, the administrator can confirm a list of work progress and corresponding proposals for each administered site.

The information indicating the work progress and corresponding proposals for each administered site is generated by the cloud service on the cloud server 50 using the health information of each worker, based on the vital data obtained from the health watch 10 and the like, and information acquired from the other systems 60, such as an attendance management system and a work process management system.

The information indicating proposals may, for example, be generated by installing an artificial intelligence function in the cloud service.

The system administrator screen cl2 illustrated in FIG. 7 is a supervision screen referred to by the administrator to supervise the status of workers in the administered site. The system administrator screen cl2 is, for example, displayed when the administrator selects one of the work areas displayed on the system administrator screen cl1 displayed in FIG. 6 (such as “work site A”, “work site B”, or “work site C”). The administrator makes the selection by, for example, moving the cursor cs1 near the position of the work area and clicking the mouse in the case of a PC, or by tapping the area in the case of a smartphone.

As illustrated, a map of the work area (for example, work site A), position information of each worker, weather information such as the temperature and the weather, vital data for each worker (information such as physical condition), a key performance indicator (KPI) that is a numerical indicator the administrator needs to manage in terms of work performance, and the like are displayed on the system administrator screen cl2.

As illustrated, human-shaped icons ic2 indicating the current position of each worker are displayed on the map of the work area in the system administrator screen cl2. The position of the icons ic2 on the map of the work area are continually updated in response to actual movement of the workers in the work area. The position of each icon ic2 on the map of the work area is, for example, updated based on position information included in the vital data that the cloud server 50 periodically acquires. The position information indicates the current position measured by a receiver, installed in the health watch 10 (or the smartphone 20), that receives radio waves of a GNSS, such as GPS or GLONASS.

In the system administrator screen cl2, the icons ic2 displayed on the map of the work area to indicate the current position of the workers are displayed in different colors in accordance with the physical condition or the like of the workers. For example, a human-shaped icon ic2 indicating a worker with no problem in his physical condition is displayed in green, whereas a human-shaped icon ic2 indicating a worker with a problem in his physical condition or the like, based on criteria described below, is displayed in yellow, red, or the like in accordance with the severity of the problem.

When the cursor cs1 is positioned near the area where an icon ic2 is displayed on the map of the work area on the system administrator screen cl2, a detailed information window image md1 pops up, as illustrated in FIG. 7, indicating detailed information related to the worker corresponding to the icon ic2.

The detailed information window image md1 includes information such as a face photograph of the worker, various vital data (such as body temperature, pulse rate, heart rate, blood pressure, amount of exercise, posture, acceleration, and position information), work start time, work end time, and contact information (such as the telephone number and e-mail address of the smartphone 20).

The display color of the icon ic2 may, for example, be determined in accordance with the aforementioned vital data. For example, if a worker satisfies any of the following conditions (abnormal worker), the display color of the icon ic2 indicating the abnormal worker becomes red to alert the administrator: a heart rate of less than 50 or more than 120 beats per minute, a blood pressure of less than 80 mmHg or more than 160 mmHg, an amount of exercise (number of steps) exceeding 12,000 steps, a body temperature exceeding 37°, or a posture remaining “sideways” for a certain time. The aforementioned reference values for changing the display color of the icon ic2 are preferably settable freely by the administrator.

When an abnormal worker is present, the cloud service on the cloud server 50, for example, transmits a message indicating the presence of an abnormal worker to an application installed on the smartphone 20 of the administrator, to the e-mail address of the administrator, or the like.

A button image (not illustrated) depicting the text “end” and a button image (not illustrated) depicting the text “call”, for example, are included in the detailed information window image md1 indicating the detailed information of the worker corresponding to the abnormal worker. When the administrator positions the cursor cs1 near the area where the button image (not illustrated) depicting the text “end” is displayed and clicks the mouse, for example, various displays indicating the abnormal state are updated to display a normal state. The display color of the icon ic2 indicating the abnormal worker, for example, is updated from red to green.

When the administrator positions the cursor cs1 near the area where the button image (not illustrated) depicting the text “call” is displayed and clicks the mouse, for example, the mobile phone number of the smartphone 20 of the corresponding worker is called. The administrator can thereby promptly contact the worker corresponding to the abnormal worker and confirm the situation.

If the button image (not illustrated) depicting the text “end” or the button image (not illustrated) depicting the text “call” is clicked, the time of the click is recorded on the cloud service as a confirmation time indicating the time that the administrator confirmed notification of the abnormal state.

KPI, which is numerical indicator the administrator needs to manage in terms of work performance, and the like are displayed in the area to the left in the system administrator screen cl2, as illustrated in FIG. 7. The number of work warnings for each type of work warning occurring on the day, for example, is displayed as a pie chart ch1 in the system administrator screen cl2, as illustrated. The number of abnormal workers (such as the number of absent workers and the number of injured workers) in the day, for example, is displayed in the system administrator screen cl2, as illustrated.

The administrator can confirm the status of workers, numerical indicators such as KPI, and the like in each administered site in real time with the system administrator screen cl2.

[Example of Functions for Managers]

The cloud service operating on the cloud server 50 not only provides administrators (such as site supervisors or remote administrators) with information such as the status of workers in the work area, the work progress, and the like but also has a function to provide managers with information.

For example, every week or month, the cloud service automatically generates a performance report with information related to health management and attendance management of workers based on information, such as vital data, obtained by the vital data collection system 1 and transmits the performance report to the e-mail address of a manager. The performance report may include not only information based on quantitative data, such as the vital data, but also suggestions for improving the way of working at the site, recommendations for social insurance, and the like.

The manager can, for example, use the information obtained by the performance report for personnel evaluation of workers and supervisors or for implementing measures such as paying rewards to units that achieve a safe work environment. The manager can also provide the information as part of compliance during an external audit.

[Operations of Communication Converter Router]

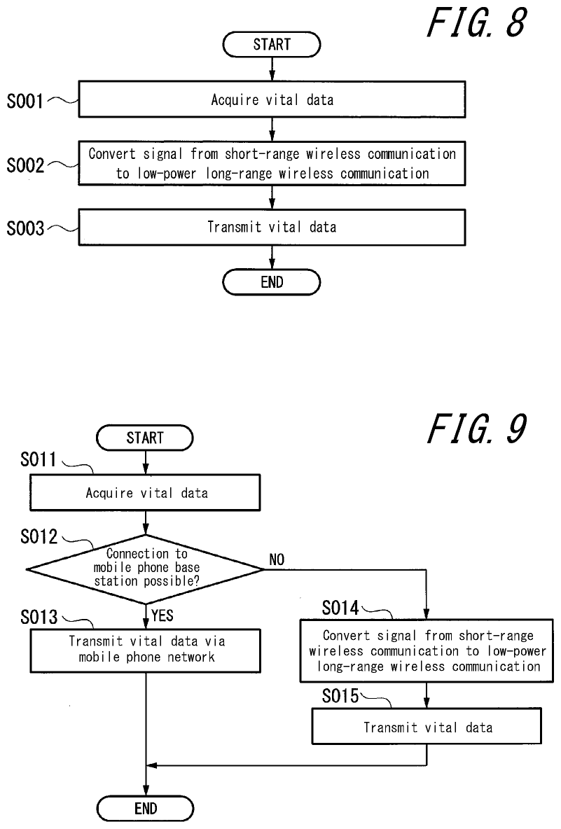

FIG. 8 is a flowchart illustrating an example of operations of the communication converter router 30 of the vital data collection system 1 according to an embodiment. This flowchart starts when vital data is transmitted from the smartphone 20 via the short-range wireless communication path 26 to the communication converter router 30.

(Step S001) The wireless A communication interface 305 of the communication converter router 30 acquires the vital data from the smartphone 20 via the short-range wireless communication path 26 and the wireless A antenna 301. The wireless A communication interface 305 outputs the vital data, based on the acquired short-range wireless communication signal, to the communication converter 307. Subsequently, the processing proceeds to step S002.

(Step S002) The communication converter 307 of the communication converter router 30 acquires vital data based on the short-range wireless communication signal outputted by the wireless A communication interface 305. The communication converter 307 converts the vital data based on the acquired short-range wireless communication signal to vital data based on a low-power long-range wireless communication signal. The communication converter 307 outputs the converted vital data based on a low-power long-range wireless communication signal to the wireless B communication interface 306. Subsequently, the processing proceeds to step S003.

(Step S003) The wireless B communication interface 306 of the communication converter router 30 acquires vital data based on the low-power long-range wireless communication signal outputted by the communication converter 307. The wireless B communication interface 306 transmits vital data based on the acquired low-power long-range wireless communication signal to the gateway 40 via the wireless B antenna 302 and the low-power long-range wireless communication path 35.

This ends the processing of the flowchart.

### Modification to Embodiment

A modification to the above embodiment is described below.

In the above embodiment, the vital data indicating the measured values measured by the health watch 10 is transmitted to the cloud server 50 via the smartphone 20 when the mobile phone network 25 is useable and is transmitted to the cloud server 50 via the smartphone 20, the communication converter router 30, and the gateway 40 when the mobile phone network 25 is not usable. With this configuration, the worker can use a known (commercial) smartphone 20. This can reduce the costs for devices carried by workers.

With the above configuration, however, the worker needs to work while both wearing the health watch 10, the wireless mounted sensor 11, and the like and carrying the smartphone 20 and the communication converter router 30. Requiring the worker to carry such a large number of devices could, in some cases, impede the work. Therefore, a configuration may be adopted such that instead of carrying both the smartphone 20 and the communication converter router 30, the worker carries one device (relay device) that combines the functions of the smartphone 20 and the functions of the communication converter router 30.

[Operations of Relay Device]

The operations of the aforementioned relay device (not illustrated) are now described with reference to the drawings.

FIG. 9 is a flowchart illustrating an example of operations of a relay device in the vital data collection system according to an embodiment. This flowchart begins when the vital data is transmitted to the relay device (not illustrated) from the health watch 10 and the like via the short-range wireless communication path 15.

(Step S011) The relay device (not illustrated) acquires the vital data in the short-range wireless communication signal from the health watch 10 and the like via the short-range wireless communication path 15. Subsequently, the processing proceeds to step S012.

(Step S012) The processing proceeds to step S013 when the relay device (not illustrated) can communicatively connect to a mobile phone base station. Otherwise, i.e. when communicative connection to a mobile phone base station is not possible, the processing proceeds to step S014.

(Step S013) The relay device (not illustrated) transmits the vital data acquired in step S011 to the cloud server via the mobile phone network 25. This ends the processing of the flowchart.

(Step S014) The relay device (not illustrated) converts the vital data based on the short-range wireless communication signal as acquired in step S011 to vital data based on a low-power long-range wireless communication signal. Subsequently, the processing proceeds to step S015.

(Step S015) The relay device (not illustrated) outputs the vital data based on the low-power long-range wireless communication signal converted in step S014 to the gateway 40 via the low-power long-range wireless communication path 35.

This ends the processing of the flowchart.

As described above, a vital data collection system according to an embodiment is for collecting vital data of an actor acting in a special environment in which communication via a mobile phone network is difficult. The vital data collection system includes a relay device that is carried by the actor, acquires vital data of the actor measured by a sensor, and transmits the acquired vital data by low-power long-range wireless communication, and a collection server that collects the vital data transmitted from the relay device.

With this configuration, the vital data collection system 1 can transmit data using commercially available terminals (such as a smartphone and a sensor) while reducing costs, even in a special environment.

The vital data collection system 1 according to an embodiment can reduce the number of relay devices and efficiently collect vital data on the cloud server 50 by use of the inexpensive, portable-sized lightweight communication converter router 30 when out of range of radio waves of a mobile phone in a special environment, such as a wide work area that is often easily affected by the weather or the like. The communication converter router 30 can acquire vital data from a commercial smartphone 20 by wireless communication that is standard on the smartphone 20 (such as Bluetooth® or Wi-Fi®) and can output the vital data after conversion to LoRa®, which is capable of long-range wireless transmission.

The present disclosure is not limited to the above-described embodiments. For example, the present disclosure may be applied to health management, attendance management, and process management not only for workers working in a plant but also for people in various other environments. For example, the present disclosure can be applied to open-pit mining in a mine, tunnel work, work on the open sea, mountain climbing, rescue activities, and construction site work.

The vital data collection system 1 according to the above-described embodiment can be configured by cloud computing and is therefore easily adaptable to various types of work and flexibly adaptable to changes in scalability, from use by small to large groups (such as 1,000 people or more).

The vital data collection system 1 can also be introduced while keeping the initial investment down. Furthermore, since the vital data collection system 1 can be configured by cloud computing, it is easy to link with another work system via an application programming interface (API) to further improve work efficiency.

During construction site work, for example, the construction work schedule is set in advance, workers are assigned based on the schedule, and an increase in overall efficiency, including attendance management and the like, is desired. The vital data collection system 1 can recommend appropriate work through vital sensing of workers, for example. Furthermore, the vital data collection system 1 can, for example, propose appropriate work process management to an administrator through an understanding of the physical condition of workers. The vital data collection system 1 can also, for example, provide a manager with support for compliance measures based on appropriate work management of employees and workers of subcontracting companies.

The vital data of workers can be anonymized in the vital data collection system 1 according to the present disclosure to allow a manager to use the vital data collection system 1 in a business that provides healthcare device manufacturers, insurance companies, and the like with anonymized vital data for a fee. As a result, healthcare device manufacturers can, for example, thereby improve the functionality of healthcare devices, and insurance companies can, for example, develop new workers' compensation insurance products for workers in special environments.

A portion or all of the vital data collection system 1 in the above-described embodiment may be implemented by a computer. In this case, a portion or all of a program for implementing these control functions may be recorded on a computer-readable recording medium, and the control functions may be implemented by a computer system reading and executing the program recorded on the recording medium.

As referred to here, the “computer system” is a computer system built into the vital data collection system 1 and includes an OS and hardware such as peripheral devices. The “computer-readable recording medium” referred to here is a storage apparatus such as a portable medium, e.g. a flexible disc, magneto-optical disc, ROM, or CD-ROM, or a hard disk or the like built into the computer system.

Furthermore, the “computer-readable recording medium” may include media that dynamically store a program for a short time, such as the communication line in the case of transmitting the program over a network such as the Internet or a communication line such as a telephone line. Media storing the program for a certain time, such as the volatile memory in the computer system that acts as the server or client in this case, may also be included. The aforementioned program may be for implementing a portion of the above-described functions and may implement the above-described functions in combination with a program already recorded on the computer system.

A portion or all of the vital data collection system 1 in the above-described embodiment may be implemented as an integrated circuit, such as a large scale integration (LSI). The functional blocks of the vital data collection system 1 may be individually formed as processors, or a portion or all of the functional blocks may be integrated and formed as a processor. The method of circuit integration is not limited to LSI. Implementation as a dedicated circuit or a general-purpose processor is also possible. Furthermore, if circuit integration technology that replaces LSI emerges through advances in semiconductor technology, this technology may be used for circuit integration.

### REFERENCE SIGNS LIST

1 Vital data collection system

10 Health watch

11 Wireless mounted sensor

15 Short-range wireless communication path

20 Smartphone

25 Mobile phone network

26 Short-range wireless communication path

30 Communication converter router

35 Low-power long-range wireless communication path

40 Gateway

45 Mobile phone network

50 Cloud server

55 Communication network

60 Other system

301 Wireless A antenna

302 Wireless B antenna

303 Lithium ion battery

304 Charging unit

305 Wireless A communication interface

306 Wireless B communication interface

307 Communication converter

1. A vital data collection system for collecting vital data of an actor acting in an environment not reached or not easily reached by radio waves from a mobile phone base station, the vital data collection system comprising:
a sensor to be carried by the actor and configured to measure vital data of the actor;
a relay device to be carried by the actor and configured to acquire the vital data of the actor measured by the sensor and to transmit the acquired vital data by low-power long-range wireless communication; and
a collection server configured to collect, via a network, the vital data transmitted from the relay device,
wherein the relay device comprises a first relay device and a second relay device to be carried by the actor;
wherein the first relay device is configured to acquire the vital data measured by the sensor, transmit the acquired vital data to the collection server via the mobile phone base station when wireless communication with the mobile phone base station is possible, and transmit the acquired vital data to the second relay device when wireless communication with the mobile phone base station is not possible; and
wherein the second relay device is configured to transmit, to the collection server by the low-power long-range wireless communication, the vital data transmitted from the first relay device.

2. (canceled)

3. The vital data collection system of claim 1,
wherein the first relay device is configured to transmit the acquired vital data by short-range wireless communication when wireless communication with the mobile phone base station is not possible; and
wherein the second relay device is configured to receive the vital data transmitted from the first relay device by the short-range wireless communication.

4. The vital data collection system of claim 1,
wherein the relay device is configured to acquire, and transmit by the low-power long-range wireless communication, at least one of position information of the sensor, time information, and identification information of the actor or the sensor in addition to the vital data; and
wherein the collection server is configured to collect the vital data transmitted from the relay device and at least one of the position information of the sensor, the time information, and the identification information of the actor or the sensor.

5. The vital data collection system of claim 1, wherein the low-power long-range wireless communication is wireless communication conforming to a LoRa® communication standard.

6-8. (canceled)

9. A vital data relay system comprising:
a first relay device and a second relay device to be carried by an actor acting in an environment not reached or not easily reached by radio waves from a mobile phone base station;
wherein the first relay device is configured to acquire vital data measured by a sensor, transmit the acquired vital data via the mobile phone base station when wireless communication with the mobile phone base station is possible, and transmit the acquired vital data to the second relay device when wireless communication with the mobile phone base station is not possible; and
wherein the second relay device is configured to transmit, by low-power long-range wireless communication, the vital data transmitted from the first relay device.

10. The vital data relay system of claim 9,
wherein the first relay device is configured to transmit the acquired vital data by short-range wireless communication when wireless communication with the mobile phone base station is not possible; and
wherein the second relay device is configured to receive the vital data transmitted from the first relay device by the short-range wireless communication.

11. The vital data relay system of claim 9, wherein the first relay device and the second relay device are configured to transmit at least one of position information of the sensor, time information, and identification information of the actor or the sensor in addition to the vital data.

12. A vital data collection method for collecting vital data of an actor acting in an environment not reached or not easily reached by radio waves from a mobile phone base station, the vital data collection method comprising:
measuring vital data of the actor, using a sensor carried by the actor;
acquiring, using a relay device carried by the actor, the vital data of the actor measured by the sensor;
performing first transmission, using the relay device, to transmit the acquired vital data to a collection server by low-power long-range wireless communication when wireless communication with the mobile phone base station is impossible;
performing second transmission, using the relay device, to transmit the acquired vital data to the collection server via the mobile phone base station when wireless communication with the mobile phone base station is possible; and
collecting via a network, using the collection server, the vital data transmitted from the relay device.

13. (canceled)

14. The vital data collection method of claim 12,
wherein the performing first transmission and the performing second transmission include transmitting the vital data acquired in the acquiring and at least one of position information of the sensor, time information, and identification information of the actor or the sensor; and
wherein the collecting includes collecting the vital data transmitted from the relay device and at least one of the position information of the sensor, the time information, and the identification information of the actor or the sensor.
